(19) United States
(12) Patent Application Publication
(10) Pub. No.: US 2011/0171294 A1
(43) Pub. Date: Jul. 14, 2011

(54) Enzyme composition and application thereof in the treatment of pancreatic insufficiency

(75) Inventors: Luppo Edens, Rotterdam (NL); Andre Leonardus De Roos, Delft (NL)

(73) Assignee: DSM IP ASSETS B.V., HEERLEN (NL)

(21) Appl. No.: 13/119,562

(22) PCT Filed: Sep. 28, 2009
(86) PCT No.: PCT/EP2009/062488

§ 371 Date: Mar. 17, 2011

(30) Foreign Application Priority Data
Sep. 30, 2008 (EP) 08165510.2

(51) Int. Cl. A61K 9/48; A61K 38/48; A61P 1/18; C12N 9/50; A61K 9/20; A61K 9/16; A61P 1/14

(52) U.S. Cl. 424/451; 424/94.63; 435/219; 424/464; 424/499

(57) ABSTRACT

The present invention relates to a composition of at least one protease and a mode of application for treating patients suffering from pancreatic enzyme insufficiency, pancreatitis or cystic fibrosis. The composition of enzymes comprises at least one protease which has a pH optimum below 5.0 and wherein said protease is further active in the presence of pepsin. In a preferred embodiment, said protease is of microbial origin.

### FIELD OF THE INVENTION

The present invention relates to a composition of at least one protease and a mode of application for treating patients suffering from pancreatic enzyme insufficiency, pancreatitis or cystic fibrosis. The composition of enzymes comprises at least one protease with a pH optimum below pH 5.0 and wherein said protease is further active in the presence of pepsin. In a preferred embodiment, said protease is of microbial origin.

### BACKGROUND OF THE INVENTION

Food compositions typically comprise proteins, carbohydrates, hemicelluloses, fats and phospholipids which during digestion are mechanically and enzymatically degraded to into smaller molecules that can be absorbed into the blood via the intestinal wall. To facilitate this degradation process, the alimentary canal of humans is a sequence of different compartments. Food is ingested and after swallowing, it reaches the stomach where it is mixed with acid and the endoprotease pepsin. Typical residence times of solid food in the stomach range from 30 minutes to a few hours. Occasional opening of the pyloris allows the acidified and partly hydrolysed food to flow into the small intestine. In the first part of the small intestine i.e. in the duodenum, bile as well as pancreatic juice are added. The pancreatic juice contains bicarbonate to partly neutralize the stomach contents. The pancreatic juice also contains an additional set of proteases, i.e. the endoproteases trypsin, chymotrypsin and elastase as well as the carboxypeptidases A and B to further degrade the peptides and polypeptides formed by the pepsin in the stomach. After the duodenum, the digest reaches the jejunum. Together with the duodenum, the jejunum presents the major site for protein absorption in the gastrointestinal tract. This absorption process involves a further proteolytic breakdown of the dietary proteins by different proteases such as amino- and carboxypeptidases or di- and tripeptidyl peptidases. The latter proteolytic hydrolysis reactions are accompanied by a facilitated transport over the intestinal wall so that small peptides as well as free amino acids end up in the blood circulation. The last part of the small intestine is formed by the ileum, after which the digest enters the large intestine (colon). In the colon, there is an intensive fermentation but there is no appreciable absorption of amino acids or peptides.

Thus digestion of food in the intestinal tract is mediated by enzymes which are amongst others secreted by the pancreas. Besides different proteases these exocrine pancreatic enzymes comprise of lipases (digestion of fats into glycerol and free fatty acids), phospholipases (digestion of phospholipids into a lysophospholipid and fatty acid) and amylases (digestion of carbohydrates into dextrins, disaccharides and free monosugars). Malfunctioning of the pancreas or failure to deliver the pancreatic enzyme products into the intestine as described for diseases such as pancreatitis, cystic fibrosis or pancreatic cancer, leads to clinical manifestations such as abdominal cramping, diarrhea, bloating and serious weight loss. Because the stomach and the pancreas form separate parts of the alimentary canal of humans and the stomach is located upstream of the pancreas, the protease (pepsin) level in the stomach of a person suffering from pancreatic enzyme insufficiency is usually normal.

For decades oral administration of porcine pancreatin (a preparation incorporating the major pancreatic enzyme products) is the standard therapy to support the digestion of food in pancreatic patients. As the pancreatin preparation is taken orally, the product will pass the stomach before entering into the duodenum. However, the pancreatic enzymes are destined to work under the near neutral conditions of the duodenum and jejunum. It is therefore not surprising that, due to the long hold up period (up to 2 hours) under the acidic conditions in the stomach, the enzymatic activities present in pancreatin preparations are gradually denatured so that the enzymatic activities present are substantially diminished. To protect the enzymatic activities against such a denaturation, the pancreatin preparation can be protected by a so-called enteric coating: a coating that stays intact under acid conditions but gradually dissolves under the near neutral pH conditions in the intestine hereby releasing the active enzymes. Unfortunately, the latter process is far from ideal because incomplete protection results in a poor enzymatic stability and an incomplete or relatively slow dissolution of the protective coating results in an incomplete release of the enzymatic activities present. Although the protective enteric coating has improved the efficacy of the oral enzyme administration, the amounts of pancreatin needed remain significant (J. H. Meyer, in Pancreatic Enzymes in Health and Disease, P. G. Lankisch, ed., p. 71-88 (1991)). The prior art describes various routes towards minimising loss of enzymatic activities resulting from stomach passage. For example, WO2001/062280 describes an enzyme composition of protease, lipase and amylase in cross-linked crystalline formulations in order to prevent the enzymes from acidic denaturation in the stomach. U.S. Pat. No. 6,051,220 describes the use of acid stable fungal amylase and acid stable lipase for the treatment of clinical conditions associated with an inadequate digestive capacity such as exocrine pancreas insufficiency.

The prior art also commonly refers to the use of proteases to replace the pancreatic endoproteases trypsin or chymotrypsin. Typical examples of such prior art proteases are the plant proteases papain (EC3.4.22.2) and bromelain (EC3.4.22.33). According to the Springer Handbook of Enzymes (second edition, volume 7; ISBN 3-540-43013-X) papain and bromelain are maximally active between pH 5 to 8.0. These pH ranges clearly indicate that the full hydrolytic activity of these enzymes will be deployed in the intestine rather than in the stomach. However, these pH optima also imply that passage of the acid stomach, will have a negative impact on the activity of these proteases. Although the pH profile of these plant endoproteases nicely mimics the pH profiles of the pancreatic endoproteases trypsin and chymotrypsin, their proteolytic activity is far from ideal for an orally taken digestion aid. In the first place it is questionable how much of their activity survives a hold-up of one to two hours under the very acidic pH conditions prevalent in the stomach. In the second place it is known that, once in the duodenum or jejunum, any remaining enzymatic activity of endoproteases such as papain and bromelain will affect the integrity and thus the activity of exogenously added other digestive enzymes such as (microbial) alpha-amylases, lipases and phospholipases.

Furthermore there are multiple publications which deal with the use of all kinds of mixtures comprising lipases, amylases and proteases from various sources. For example, US patent application 20040057944 describes the use of a concentrated lipase of Rhizopus delemar, a neutral protease of Aspergillus melleus and an amylase of Aspergillus oryzae. US patent application 20080199448 also describes the use of a lipase, a protease and an amylase from a variety of microorganisms and plant material.

### DESCRIPTION OF THE FIGURES

FIG. 1: The pH profiles of the tripeptidyl amino peptidases encoded by genes having sequence 10 (dotted line) and 12 (solid line) of A. niger as specified in WO 02/068623 and as determined on the synthetic substrate Ala-Ala-Phe-pNA.

FIG. 2: The pH profile of the proline-specific endoprotease as specified in EP 0 522 428 and as determined on the synthetic substrate Z-Gly-Pro-pNA.

FIG. 3: The pH profile of the carboxypeptidase CPD-I as determined on the synthetic substrate FA-Phe-Ala.

### SUMMARY OF THE INVENTION

Although multiple (commercial) compositions for treating pancreatic enzymatic insufficiency are available, all of them have one or multiple drawbacks. For example these enzymes are commonly provided as crude enzyme mixtures so that large amounts are required to reach an acceptable efficacy. Additionally, the pancreatic enzymes are inherently instable in the stomach, so expensive enteric coatings are required. The untimely and incomplete release of the enzymatic activity from such coated preparations imply that part of the enzymatic activity is lost resulting in partial instead of complete degradation of the food substances complicating their transport over the intestinal wall. Finally, the amylolytic and lipolytic enzymatic activity present in these preparations run the risk of being degraded by the proteases active in the intestines.

It is an object of the invention to provide an enzyme composition which lacks one or multiple of the drawbacks mentioned.

The inventors found that the at least one protease which is active in the stomach, i.e. a protease which is capable of digesting proteins and peptides under acidic conditions can be used, in a composition for treating pancreatic enzyme insufficiency, pancreatitis or cystic fibrosis. This protease acts together with pepsin naturally present in the stomach. Preferably said at least one protease is inactive or less active under the higher pH conditions prevalent in the duodenum and beyond. Because said protease is active in the acid surrounding of the stomach, an expensive enteric coating is not required, smaller amounts of said protease are used and hence smaller pharmaceutical products (such as but not limited to tablets) have to be consumed by a patient in need of pancreatic enzymatic insufficiency treatment. Another advantage is that food will have in general a residence time of 30 to 120 minutes in the stomach and the protease or the composition of the invention has sufficient time to hydrolyze the food. The nature of said protease is such that, in conjunction with the stomach protease pepsin, it can degrade dietary proteins into peptides small enough to pass the intestinal wall. The protease is not a pepsin, which is already present in the stomach.

The present invention relates to a pharmaceutical or nutritional composition for the treatment of pancreatic enzyme insufficiency, pancreatitis or cystic fibrosis comprising at least one protease with a pH optimum below pH 5.0 and wherein said protease is active in the presence of pepsin. Preferably said at least one protease is a tripeptidase more preferably a tri-peptide-aminopeptidase. Optionally the pharmaceutical or nutritional composition may comprise a proline specific endoprotease (PSE or EndoPro) and/or a carboxypeptidase, which are preferably acid-stable and/or active in the presence of pepsin.

Although a mix of multiple proteases with an acidic pH optimum can be combined, also only one active ingredient can be used, i.e. one protease without additional enzymatic activities (essentially pure).

According to a preferred embodiment of the invention the proteolytic activity of the pancreatic enzyme supplement preferably comprises a tripeptidase, more preferably a tripeptidyl aminopeptidase (TPAP; EC 3.4.14.9) and/or a mixture of tripeptidases. Such tripeptidases are defined as enzymes capable of releasing tripeptides from a polypeptide, either from the N-terminal side of the polypeptide hereby encompassing the so-called tripeptidyl-aminopeptidases or from the C-terminal side of the polypeptide hereby encompassing the so-called peptidyl-tripeptidases. Optionally the mixture may comprise an acid stable proline specific endoprotease (PSE or EndoPro) and/or an acid stable carboxypeptidase.

A major advantage of the present invention is that a protease preferably an acid tripeptidase will be active under the acid pH conditions in the stomach but not under the higher pH conditions in the duodenum and beyond, so that the auxiliary alpha-amylase, lipase and phospholipase activities (which can, for example, be supplied via commercial available compositions) will not be unfavorably affected.

Still another aspect of the at least one selected acid protease is that it completes the proteolytic action of the endoprotease pepsin (EC 3.4.23.1), secreted into the gastric juice of vertebrates, into small peptides and free amino acids which can be readily transported over the intestinal wall to end up in the blood circulation.

All proteolytic enzymes used in the composition of the invention are preferably from fungal origin, more preferably obtained from Aspergilli, and diverge from endoproteases such as the plant derived papain and bromelain in being a tripeptidase preferably a tri-peptide-aminopeptidase (TPAP), optionally supplemented with a proline-specific endoprotease and/or a carboxy-exoprotease.

In yet another embodiment, the invention provides a composition comprising at least one serine-type protease with an acidic pH optimum and wherein said protease is further active in the presence of pepsin.

### DETAILED DESCRIPTION OF THE INVENTION

In a first embodiment, the invention provides a composition for the treatment of pancreatic enzyme insufficiency, pancreatitis or cystic fibrosis comprising at least one protease which has its pH optimum below pH 5.0 (i.e. with a pH optimum below pH 5.0) and said protease is active in the presence of pepsin. In another embodiment, the invention provides a composition comprising at least one protease which is active under acidic conditions, preferably active in the stomach, and said protease is active in the presence of pepsin. Such proteases are extremely useful to digest proteins in the acidic environment of the stomach. Preferably said at least one protease having a pH optimum below 5 and/or being active under acidic contions like the stomach, is a serine-type protease. The composition according to the invention is preferably a composition for (at least in part) improving the absorption and digestion of food. This may relate to in vivo absorption and/or digestion, i.e. in a mammal, or to in vitro absorption and/or digestion, i.e. in a model system of which multiple are available. In its simplest form the model system can be an in vitro system held for fixed periods at a pH value that is gradually lowered to pH 2.0. The presence of the gastric endoprotease pepsin is mimicked by simply adding the purified pepsin enzyme to the incubation mixture. In a more sophisticated approach, the passage of food through the gastrointestinal tract can be simulated in dynamic gastrointestinal in vitro models. In such models the successive dynamic processes in the stomach and in the small intestine are simulated using validated procedures (Minekus et al, ATLA 1995, 23, 197-209; Larsson et al, J Sci Food Agic 1997, 74, 99-106).

As will be explained in more detail later on, the composition of the invention is suitable for treating pancreatic enzyme insufficiency.

Said composition is a pharmaceutical, nutraceutical or nutritional composition comprising said at least one protease which has its pH optimum below pH 5.0 and said protease is further active in the presence of pepsin, as an active ingredient and optionally comprising at least one (but typically more than one, i.e. multiple) excipients.

According to another aspect of the invention said composition is a pharmaceutical or nutraceutical composition for the treatment of pancreatic enzyme insufficiency, pancreatitis or cystic fibrosis comprising said at least one protease which has a pH optimum below pH 5 and/or is active under acidic conditions, preferably active in the stomach said protease is further active in the presence of pepsin, as an active ingredient and optionally comprising at least one (but typically more than one, i.e. multiple) excipients. For use in an in vitro model system a composition according to the invention does not necessarily comprise excipients and hence in such a case the term composition is more applicable.

The skilled person is very well capable of determining whether a protease has its pH optimum below pH 5.0 by contacting said at least one protease with a biological or synthetic substrate of said protease at a fixed temperature (preferably 37 degrees C.) under various pH values by using appropriate buffer systems, allowing said protease sufficient amount of time, for example 10 minutes, to interact with said substrate and determining at which pH value the highest enzyme activity can be recorded. Examples of such pH optimum determinations using synthetic peptide substrates are provided in Example 1 of the present application. To establish whether said at least one protease is active in the presence of pepsin the skilled person preferably preincubates said at least one protease with pepsin at various acidic pH values and subsequently the thus treated protease is incubated with the one of the mentioned substrates. Alternatively pepsin, said at least one protease and substrate are jointly incubated at various acidic pH values after which the residual activity of said at least one protease is established. An example of the latter approach is provided in Example 2 of the present application.

As will be explained in more detail later on, a composition according to the invention preferably comprises one, two or even more than two proteases which all have their pH optimum below pH 5.0 and wherein said protease is further active in the presence of pepsin. The present invention therefore also relates to the use of a mixture of proteases that have their pH optimum below pH 5.0 and are active in the presence of pepsin. In yet another preferred embodiment, the invention provides a composition with only one type of active ingredient, i.e. one protease with a pH optimum below 5 and further being active in the presence of pepsin. Preferably, said protease is a serine-type protease.

Preferably, said at least one protease is inactive or less active at a pH of 6.0 or higher such as prevailing in the gastro-intestinal tract downstream of the stomach (compared to its activity at pH 4.0). In percentages, the used protease has an activity at pH 6.0 or higher of less than 50%, preferably less than 40% and even more preferably less than 30% compared to its activity at pH 4.0. To avoid inactivation of other enzymes (such as lipases, amylases and (more alkaline) proteases) after passage of the stomach, the activity of said at least one protease is less than 25%, 20%, 15%, 10%, 9%, 8%, 7%, 6%, 5%, 4%, 3%, 2% or 1% or even less of its activity at pH 4.0. In a preferred embodiment, the invention provides a composition comprising at least one protease which has its pH optimum below pH 5.0 and wherein said protease is further active in the presence of pepsin, wherein said protease has at pH 6.0 or higher an activity of less than 50%, preferably less than 40% and even more preferred less than 30% compared to its activity at pH 4.0. In case more than one protease is used which all have their pH optimum below pH 5.0 and wherein said protease is further active in the presence of pepsin, it is preferred that at least one of said used proteases has at pH 6.0 or higher an activity of less than 50%, preferably less than 40% and even more preferred less than 30% compared to their activity at pH 4.0.

The above described requirements for said at least one protease results in a feature which can also be described as having a pH optimum curve with declines sharply, i.e. within 2 or 1 or even 0.5 pH units from optimal (highest activity) to close to zero activity towards the near neutral pH values. The experimental part shows such pH profiles for some suitable proteases that can be used in a composition according to the invention.

An important aspect of the at least one selected acid proteases is that it completes the proteolytic action of the aspartic endoprotease pepsin in such a way that it generates in the stomach small peptides and free amino acids which can be readily transported over the intestinal wall to end up in the blood circulation. Pepsin (EC 3.4.23.1) is an endoprotease with a broad specificity capable of predigesting many different dietary proteins. A precursor of the enzyme is synthesized in the gastric mucosa and, after its activation by the acid milieu of the stomach, the enzyme represents the principle acid protease of the stomach. The enzyme is active between a pH value from below 1 to about 6 with an optimum around pH 3.5. However, only relatively large peptides form a good substrate for the enzyme. In healthy individuals these relatively large peptides formed in the stomach are converted into absorbable free amino acids and di- and tripeptides by the different proteases that are active under the near neutral pH conditions of the intestine. As in individuals suffering from pancreatic enzyme insufficiency, pancreatitis or cystic fibrosis the latter digestion process is incomplete, such absorbable free amino acids and di- and tripeptides are formed to a lesser extent or not formed at all. We have now found that upon the oral intake of the at least one selected acid protease, absorbable free amino acids and di- and tripeptides are already efficiently formed in the stomach so that even in the absence of pancreatic secretions, dietary proteins can be efficiently taken up and metabolized.

To be useful for the pharmaceutical or nutritional treatment of pancreatic enzyme insufficiency, pancreatitis or cystic fibrosis described here, said at least one protease must preferably meet a number of strict economical and legislative criteria. To meet the legislative criteria the enzyme should preferably be obtained from an unsuspect source, for example a food-grade microorganism. To meet the economical criteria, the enzyme should preferably be secreted by a microorganism, producible in high yields and exhibit a number of biochemical characteristics such as a long term stability under industrial processing conditions. To minimize the risks of microbial infections under such non-sterile conditions, industrial processing often employs acidic pH conditions and a temperature of 50 degrees C. or higher. A protease used in the present invention advantageously meets these demands.

The internationally recognized schemes for the classification and nomenclature of all enzymes from IUMB include proteases. The updated IUMB text for protease EC numbers can be found at the internet site: http://www.chem.qmw/ac.uk/iubmb/enzyme/EC3/4/11/. In this system enzymes are defined by the fact that they catalyze a single reaction. This has the important implication that several different proteins are all described as the same enzyme, and a protein that catalyses more than one reaction is treated as more than one enzyme.

The system categorises the proteases into endo- and exoproteases. Endoproteases are those enzymes that hydrolyze internal peptide bonds of proteins and exoproteases are those enzymes that hydrolyze peptide bonds adjacent to a terminal α*amino group (so-called “aminopeptidases”), or a peptide bond between the terminal carboxyl group and the penultimate amino acid (so-called “carboxypeptidases”). The endoproteases are divided into sub-subclasses on the basis of catalytic mechanism. There are sub-subclasses of serine endoproteases (EC 3.4.21), cysteine endoproteases (EC 3.4.22), aspartic endoproteases (EC 3.4.23), metalloendoproteases (EC 3.4.24) and threonine endoproteases (EC 3.4.25).

Tripeptidyl aminopeptidases (TPAP's) are exoenzymes that can release tripeptides from the N-terminus of an oligopeptide. Little is known on enzymes that can release tripeptides from the oligopeptide's carboxyterminus (“tripeptidyl carboxypeptidases or peptidyl-tripeptidases”).

Tripeptidyl aminopeptidases (EC 3.4.14) have been isolated from mammalian as well as plant sources. Micro-organisms from which tripeptidylpeptidases have been isolated are for example Streptomyces species (JP08308565, WO 95/17512 and U.S. Pat. No. 5,856,166), Porphyromonas gingivalis (WO 00/52147), Dictyostelium discoidum and Aspergillus species (WO 96/14404). To date, the occurrence of tripeptidyl carboxypeptidases (EC 3.4.15) has been demonstrated in mammalian cells and in the microorganism Clostridium histolyticum only.

In a preferred embodiment, the invention provides a composition comprising at least one protease which has its pH optimum below pH 5.0 and wherein said protease is further active in the presence of pepsin, wherein said protease is a tripeptidase. Said tripeptidase may be a tripeptidylaminopeptidase or a peptidyl-tripeptidase.

Peptidases especially suitable in the present invention are described in WO 02/068623. These enzymes are obtained from A. niger. In Table 1 of WO 02/068623 the SEQ ID numbers of the tripeptidases are given. The protein products from gene 9, 10 (=TPAP-B), 12 (=TPAP-A), 19, 26, 35 and 50 are especially useful in a composition according to the invention. Among these the protein products of gene 12 (TPAP-A) and gene 10 (TPAP-B) are of special importance because of their high production yields, excellent temperature stabilities and high specific activities.

From an economic point of view the implication of our observations is that there exists a clear need in the present process for the use of tripeptidases in high quantities and in a pure or isolated form. A preferred way of obtaining purified and isolated tripeptidases is via the overproduction using recombinant DNA techniques. A particularly preferred method is the overproduction of such tripeptidases derived from Aspergillus and a most preferred method is the overproduction of such tripeptidases from Aspergillus niger.

According to the invention several useful tripeptidases are preferably used in a pure or isolated state. A pure tripeptidase can be obtained for example by overexpression of the enzyme is a suitable transformed host micro-organism.

In the presence of the gastric endoprotease pepsin, a tripeptidase which has its pH optimum at pH 5.0 or lower and wherein said protease is further active in the presence of pepsin, is very well capable of digesting proteins into smaller parts that can be absorbed in the blood through the intestinal wall without any further protease action needed. However, the digestion process may be further improved or speeded up by using a second or even a second and a third protease. Said second or said second and said third protease preferably also has/have its pH optimum below pH 5.0 and also is/are further active in the presence of pepsin.

Therefore, the invention further provides a composition comprising at least one protease which has its pH optimum below pH 5.0 and wherein said protease is further active in the presence of pepsin, further comprising a second protease.

Preferably said second protease is a carboxypeptidase or a proline-specific endoprotease. In a preferred embodiment said second protease is a carboxypeptidase, because this type of enzyme has a pH profile which is very comparable to one of the exemplified tripeptidyl aminopeptidases (TPAP's). A composition according to the invention may further comprise a third protease. When the second protease is a carboxypeptidase, said third protease is preferably a proline-specific endoprotease and when said second protease is a proline-specific endoprotease said third protease is preferably a carboxypeptidase. As outlined above, a preferred first protease is a TPAP and hence the invention preferably provides a composition comprising a TPAP and a carboxypeptidase or a TPAP and a proline-specific endoprotease. Even more preferred said composition comprises TPAP, a proline-specific endoprotease and a carboxypeptidase. Preferably TPAP, proline-specific endoprotease and carboxypeptidase are all serine-type proteases.

In conjunction with prior art endoproteases, the proline-specific endoprotease is capable of extensively hydrolysing proline-rich proteins yielding relatively small peptides with a narrow size distribution. A preferred way of obtaining purified and isolated PSE is via the overproduction of such a proline-specific endoprotease using recombinant DNA techniques.

A proline-specific endoprotease (PSE) is an enzyme capable of cleaving peptides or polypeptides at the carboxy-terminal end of proline residues. Proline-specific endoproteases are widely found in animals and plants, but their presence in microorganisms appears to be limited. To date, proline-specific endoprotease have been identified in species of Aspergillus (EP 0 522 428 and WO 02/45524), Flavobacterium (EP 0 967 285), Aeromonas (J. Biochem. 113, 790-796), Xanthomonas and Bacteroides. However, only few of these proline-specific proteases exhibit acid pH optima.

A preferred way of obtaining purified and isolated PSE is via the overproduction of such a proline-specific endoprotease using recombinant DNA techniques. As many food products are acidic and long term enzyme incubations under industrial, non-sterile circumstances require acidic incubation conditions and a processing temperature of 50 degrees C. or higher to prevent microbial contamination, a more preferred method is the overproduction of an acid stable proline-specific endoprotease using recombinant DNA techniques. A particularly preferred method is the overproduction of an Aspergillus derived proline-specific endoprotease and a most preferred method is the overproduction of an Aspergillus niger derived proline-specific endopeptidase.

Carboxypeptidases are described in the prior art. For example, carboxy-peptidase CPD I (PEPG) from an Aspergillus strain is described. Examples of suitable Aspergilli are A. niger, A. oryzae and A. sojae. Preferably a serine-type carboxypeptidase belonging to enzyme class EC 3.4.16.1 is used, more preferably a non-animal derived serine-type carboxypeptidase belonging to enzyme class EC 3.4.16.1 is used, even more preferably a preferably serine-type carboxypeptidase belonging to enzyme class EC 3.4.16.1 obtained from Aspergillus is used, most preferably serine-type carboxypeptidase CPD 1 from A. niger is used. The latter enzyme has been described (Dal Degan, Ribadeau-dumas & Breddam, Appl. Environ. Microbiol (1992) 58, 2144-2152) and has been sequenced (Svendsen & Dal Degan, Bioch. Biophys. Acta (1998) 1387, 369-377).

Although a tripeptidase can be combined with another protease, it is preferred that the only active compound in said composition is a tripeptidase, such as but not limited to a TPAP.

Advantageously the enzymes are used in an isolated form and in a tripeptidase enzyme protein to carboxypeptidase/proline-specific endoprotease enzyme protein weight ratio range between 1:0.01 and 1:1.

A polypeptide used in the present invention which has tripeptidase activity may be in an isolated form. As defined herein, an isolated polypeptide is an endogenously produced or a recombinant polypeptide which is essentially free from other polypeptides, and is typically at least 20% pure, preferably at least 40% pure, more preferably at least 60% pure, even more preferably at least 80% pure, still more preferably at least 90% pure, or most preferably at least 95% pure, as determined by SDS-PAGE. The polypeptide may be isolated by centrifugation, filtration (for example utrafiltration) optionally followed by chromatographic methods, or any other technique known in the art for obtaining pure proteins from crude solutions. It will be understood that the polypeptide may be mixed with carriers or diluents which do not interfere with the intended purpose of the polypeptide, and thus the polypeptide in this form will still be regarded as isolated. It will generally comprise the polypeptide in a preparation in which more than 10%, for example more than 20%, 30%, 40%, 50%, 80%, 90%, 95% or 99%, by weight of the proteins in the preparation is a polypeptide for use of the process of the present invention.

Although not limited to it, a composition as described above is preferably a pharmaceutical or nutritional composition suitable for oral administering, i.e. the invention preferably provides an oral composition, i.e. a composition suitable for oral intake. As a consequence, the proteases described in general above (and more specific TPAP, a proline-specific endoprotease and/or a carboxypeptidase) are preferably present in a pill, granulate, capsule or tablet and even more preferably in an acid-instable pill, granulate, capsule or tablet. When more than one protease which has its pH optimum below pH 5.0 and which is further active in the presence of pepsin are used as active ingredient, the different active ingredients may be present within one and the same (acid-instable) pill, granulate, capsule or tablet or within different (acid-instable) pills, granulates, capsules or tablets. When three active ingredients are used two of them may be combined in one (acid-instable) pill, granulate, capsule or tablet and the third one may be separate or all active ingredients can be present in one (acid-instable) pill, granulate, capsule or tablet or all active ingredients are separately formulated as a (acid-instable) pill, granulate, capsule or tablet.

The term “acid-instable” is used to describe that the pill, granulate, capsule or tablet liberates the active compound or ingredient under acidic conditions (such as the stomach), for example the pill, granulate, capsule or tablet disintegrates in an acidic environment, i.e. at a pH lower than pH 6.0 such that the active ingredients are freed from said pill, granulate, capsule or tablet and are able to perform their function in the stomach. Alternatively, the pill, granulate, capsule or tablet comprising TPAP (in a preferred embodiment as the sole active ingredient) is present in a non-enteric coated pill, granulate, capsule or tablet. Such a pill, granulate, capsule or tablet allows activity of the used protease in the stomach (after oral intake thereof). The term “acid-instable” and “non-enteric coated” are used interchangebly herein.

For preparing a pill, granulate, capsule or tablet according to the invention, the proteases according to the invention are, after fermentation recovered using known methods. Briefly, from the fermented liquid the biomass is filtered off and the resulting liquid is then ultrafiltered to concentrate and recover the enzyme containing broth. Optionally the resulting concentrate can be subjected to one or more diafiltration steps to lower its salt content. Optionally the diafiltered liquid can be subjected to chromatography to further increase the purity of the enzyme product. The final enzyme concentrate can be stabilized and used as such, e.g. to fill a capsule. To obtain the enzyme in a dry form which is preferred for the present invention, the concentrated liquid is then preferably spray dried and the resulting powder is used for the pill, granulate, capsule or tablet production.

Therefore the present invention provides a pharmaceutical or nutritional composition for the treatment of pancreatic enzyme insufficiency, pancreatitis or cystic fibrosis comprising at least one protease with a pH optimum below pH 5.0 and wherein said protease is active in the presence of pepsin, wherein said protease(s) is/are present in an acid-instable pill, granulate, capsule or tablet.

Another embodiment of the invention relates to an acid-instable pill, granulate, capsule or tablet for the treatment of pancreatic enzyme insufficiency, pancreatitis or cystic fibrosis comprising at least one protease with a pH optimum below pH 5.0 and said protease is further active in the presence of pepsin and to an acid-instable pill, granulate, capsule or tablet for the treatment of pancreatic enzyme insufficiency, pancreatitis or cystic fibrosis comprising at least one protease which is active at acidic pH, preferably in the stomach, and said protease is further active in the presence of pepsin. Said at least one protease is preferably a tripeptidase, preferably a tripeptidylaminopeptidase.

The present invention also relates to a method for preparing said acid-instable pill, granulate, capsule or tablet for the treatment of pancreatic enzyme insufficiency, pancreatitis or cystic fibrosis comprising obtaining at least one protease with a pH optimum below pH 5.0 and said protease is active in the presence of pepsin and preparing an acid-instable pill, granulate, capsule or tablet comprising the protease or a method for preparing said acid-instable pill, granulate, capsule or tablet for the treatment of pancreatic enzyme insufficiency, pancreatitis or cystic fibrosis comprising obtaining at least one protease which is active at acidic pH, preferably in the stomach, and said protease is active in the presence of pepsin and preparing an acid-instable pill, granulate, capsule or tablet comprising the protease.

According to a further aspect the present invention provides the use of at least one protease with a pH optimum below pH 5.0 or which is active at acidic pH, preferably in the stomach, and wherein said protease is further active in the presence of pepsin for the manufacture of a medicament for the treatment of pancreatic enzyme insufficiency, pancreatitis or cystic fibrosis preferably said protease is a tripeptidase, more preferably said tripeptidase is a tripeptidyl aminopeptidase or peptidyl-tripeptidase, most preferably a tripeptidyl aminopeptidase. In a preferred embodiment said at least one protease is present in an acid instable pill, granulate, capsule or tablet. More preferably said acid instable pill, granulate, capsule or tablet further comprises a proline-specific endoprotease and/or a carboxypeptidase.

Another aspect of the invention relates to a method for treating malabsorption in a mammal comprising administering to said mammal a therapeutically effective amount of the composition of the invention. Preferably said method for treating pancreatic enzyme insufficiency, pancreatitis or cystic fibrosis in a mammal comprises administering to said mammal a therapeutically effective amount of the composition of the invention. the composition of the invention can also be used in a method for supplementing enzymes in a mammal for the treatment of pancreatic enzyme insufficiency, pancreatitis or cystic fibrosis comprising administering to said mammal a therapeutically effective amount of the composition of the invention, preferably said composition is in the form of a pill, granulate, capsule or tablet for example wherein the composition is administered to said mammal with a meal, snack or shot.

A composition or dosage unit according to the invention may further be supplemented with one or more other enzymes, for example amylases, lipases, (in respect of pH, neutral to slightly alkaline) proteases and phospholipases.

Compositions comprising a lipase, an amylase and a protease are well known and are already (commercially) used in the treatment of pancreatic enzyme insufficiency. To prevent their inactivation in the acidic environment of the stomach such oral compositions are typically in the form of an enteric coated pill, granulate, capsule or tablet.

According to another embodiment of the present invention, such a (commercially available) enteric coated pill, granulate, capsule or tablet comprising a lipase, an amylase and/or a (neutral slightly alkaline) protease is combined with an acid-instable pill, granulate, capsule or tablet comprising for example TPAP and optionally a proline-specific endoprotease (or TPAP and a carboxypeptidase or TPAP and a proline-specific endoprotease and a carboxypeptidase) according to the present invention. Subsequently the present invention also provides a composition comprising at least 2 different types of enzymes: a lipase, optionally an amylase, optionally with a neutral to slightly alkaline protease, and at least one protease with a pH optimum below pH 5.0 and wherein said protease is further active in the presence of pepsin (such as but not limited to a tripeptidase). Preferably such a composition comprises at least 2 separate pills, granulates, capsules or tablets or any combination thereof. Even more preferably said acidic and pepsin stable protease (for example a tripeptidase) is present in a pill, granulate, capsule or tablet separate from the other enzymes. The other enzymes may be divided over multiple pills, granulates, capsules or tablets or can be present in one pill, granulate, capsule or tablet. Even more preferably the tripeptidase is present in an acid-instable pill, granulate, capsule or tablet and the other enzymes (such as a lipase, optionally an amylase and optionally a neutral to slightly alkaline protease) are present in one or are divided over multiple enteric coated pill(s), granulate(s), capsule(s) or tablet(s).

So the invention provides a pharmaceutical kit or kit of parts for the treatment of pancreatic enzyme insufficiency, pancreatitis or cystic fibrosis which comprises an acid-instable pill, granulate, capsule or tablet comprising for example TPAP and an enteric coated pill, granulate, capsule or tablet comprising a lipase, optionally an amylase, optionally with a neutral to slightly alkaline protease.

Commercial enteric coated pancreatine preparations are sold and can easily be obtained. Pancreatin is a preparation of porcine pancreatic enzymes. Pancreatin naturally contains protease (protein digesting), amylase (carbohydrate digesting), and lipase (fat digesting) enzymes. The different available preparations may vary in respect of the exact amount of enzymes as well as their relative ratio. For example, one commercial preparation comprises protease:amylase:lipases in a ratio of 337:94:442 USP/mg (USP=United States Pharmacopae units) respectively. Another preparation comprises Proteases 45,000 NFU, Amylases 67,500 NFU/tablet, Lipases 7050 NFU/tablet (NFU=National Formulary Unit).

Also preferred is a combination of an acid-instable pill, granulate, capsule or tablet comprising for example TPAP and a proline-specific endoprotease (or TPAP and a carboxypeptidase or TPAP and a proline-specific endoprotease and a carboxypeptidase) with an enteric coated pill, granulate, capsule or tablet comprising a lipase, a phospholipase and an amylase. Preferably said phospholipase is phospholipase A2 and even more preferably said phospholipase is a microbial phospholipase. Because the acidic and pepsin stable proteases are very efficient in the breakdown of proteins within the stomach, the commonly used proteases (having their pH optima in the near neutral to slightly alkaline region) are not longer required resulting in the above mentioned advantages of the present invention. Again, the active ingredient that needs to be protected from the acidic conditions in the stomach may be present in one or divided over multiple enteric coated pill(s), granulate(s), capsule(s) or tablet(s).

Because the used tripeptidase is very efficient, another preferred combination of additional pancreatic enzymes is devoid of proteases. Such a composition can comprise a lipase alone, or a lipase and/or a phospholipase and/or an amylase. Again, to avoid degradation in the stomach, an enteric coated pill, granulate, capsule or tablet is used for the latter compositions. Advantageously, the enzymes in the enteric coated pill are not degraded by a protease and as a consequence lower amounts of enzymes can be administered. In a preferred embodiment the invention therefore provides a kit comprising at least two different pills, granulates, capsules or tablet that differ in their enzyme compounds: a first pill, granulate, capsule or tablet comprises for example a TPAP and is acid-instable; the second pill, granulate, capsule or tablet comprises a lipase and/or an amylase and/or a phospholipase and is enteric coated. The second pill, granulate, capsule or tablet is preferably free from neutral or slightly alkaline proteases.

The skilled person is very well aware of the presence of suitable lipases, amylases, phospholipases and alkaline proteases or how to measure the presence of such enzymatic activities so that there is no need to elaborate on this further.

One or multiple enzymes present in the above described enteric coated pill, granulate, capsule or tablet may be present in a crystal form. This enhances the stability to the acidic pH of the stomach and/or its resistance to proteolytic degradation. Enzyme crystals can be obtained via methods well described in prior art.

According to the invention all individual enzymes (i.e. acidic proteases, alkaline proteases, amylases, lipases, phospholipases and so on) are preferably used in a pure or isolated state. Pure enzymes can be obtained for example by overexpression of the enzyme in a suitable transformed host micro-organism. The advantage of microbial enzymes over animal derived enzymes is their abundance of supply, their unsuspect nature and their potential Kosher status.

In yet another embodiment the invention provides an acid-instable pill, granulate, capsule or tablet comprising at least one protease with a pH optimum below pH 5.0 and wherein said protease is further active in the presence of pepsin. Preferably, said at least one protease is a tripeptidase. Said tripeptidase may be a tripeptidylaminopeptidase or a peptidyl-tripeptidase. Relevant information in respect of said tripeptidase is already described above and applies mutatis mutandis to this subject matter. In one of its preferred embodiments, the invention provides an acid-instable pill, granulate, capsule or tablet comprising one active ingredient, and wherein said one active ingredient is a protease with a pH optimum below pH 5.0 and wherein said protease is further active in the presence of pepsin.

The invention further provides an acid-instable pill, granulate, capsule or tablet comprising a proline-specific endoprotease or an acid-instable pill, granulate, capsule or tablet comprising a carboxypeptidase. Preferably one or all enzymes are combined into one acid-instable pill, granulate, capsule or tablet, i.e. an acid-instable pill, granulate, capsule or tablet comprising a tripeptidase, a proline-specific endoprotease and/or a carboxypeptidase.

The invention also provides combinations (or a kit) of the acid-instable pill, granulate, capsule or tablet according to the invention together with an enteric coated pill, granulate, capsule or tablet comprising either a lipase, a phospholipase and an amylase or a combination of one or more of such activities. Preferably said phospholipase is a microbial phospholipase and even more preferred said phospholipase is phospholipase A2. This combination or kit is very suitable for the treatment of pancreatic enzyme insufficiency, pancreatitis or cystic fibrosis.

To apply enteric coatings both solvent and aqueous processing techniques are available; see for example publications by Colorcon, West Point, Pa. or visit http://www.pharma-excipients.com/aqueous-enteric-coating-polymer.html.

A pill, granulate, capsule or tablet as described above preferably further comprises at least one excipient as an inactive ingredient, the excipient being selected from, for example, a filler, a flow agent, a colorant, a flavoring, a dissolving agent, and any combination thereof. The excipient may comprise up to 95 weight percent of an acid-instable pill, granulate, capsule or tablet.

In a preferred embodiment the active ingredient in an acid-instable pill, granulate, capsule or tablet according to the invention consists only of one or multiple acid and pepsin stable protease(s) which is/are inactive or less active at pH 6.0 or higher (compared to their activity at pH 4.0).

The preparation of an enteric coated pill, granulate, capsule or tablet is well known in the prior art and hence no elaborate information is given in this respect.

In yet another embodiment the invention provides a method for preparing an acid-instable pill, granulate, capsule or tablet as described above, comprising obtaining at least one protease having a pH optimum below pH 5.0 or being active under acidic conditions, preferably in the stomach, and wherein said protease is further active in the presence of pepsin and preparing an acid-instable pill, granulate, capsule or tablet therefrom. The experimental part describes the preparation of a tablet. Other proteases such as a proline-specific-protease or a carboxypeptidase may be included in one and the same acid-instable pill, granulate, capsule or tablet or may be divided over two or even more acid-instable pills, granulates, capsules or tablets.

A composition as described above is very useful in the treatment of pancreatic enzyme insufficiency, pancreatitis or cystic fibrosis. The invention therefore also provides the use of at least one protease with a pH optimum below pH 5.0 and wherein said protease is further active in the presence of pepsin for the manufacture of a medicament for the treatment of pancreatic enzyme insufficiency, pancreatitis or cystic fibrosis. In a preferred embodiment, said at least one protease is present in an acid instable pill, granulate, capsule or tablet. More preferably said at least one protease is TPAP optionally comprising a second acid and pepsin stable protease such as a proline-specific endoprotease or a carboxypeptidase.

In another preferred embodiment, the compositions of the invention are useful in methods for treating pancreatic enzyme insufficiency, pancreatitis or cystic fibrosis in a mammal subject, including a human suffering from cystic fibrosis. The invention thus provides a method for treating malabsorption in a mammal comprising administering to said mammal a therapeutically effective amount of a composition comprising at least one protease with a pH optimum below pH 5.0 and wherein said protease is further active in the presence of pepsin.

In an alternative embodiment, the invention provides a method for treating pancreatic enzyme insufficiency, pancreatitis or cystic fibrosis in a mammal comprising administering to said mammal a therapeutically effective amount of a composition comprising at least one protease with a pH optimum below pH 5.0 and wherein said protease is further active in the presence of pepsin. A further alternative embodiment of a method according to the invention is a method for supplementing pancreatic enzymes in a mammal comprising administering to said mammal a therapeutically effective amount of a composition comprising at least one protease with a pH optimum below pH 5.0 and wherein said protease is further active in the presence of pepsin. Any of the methods may be used to treat a pancreatic enzyme insufficiency, pancreatitis or cystic fibrosis patient.

In the context of the described method of treatments, the used composition is typically suitable for oral administration.

The preferred proteases in methods for treatment according to the invention are TPAP, optionally in combination with a proline-specific endoprotease or a carboxypeptidase (or any combination thereof). In a preferred embodiment, TPAP is the sole or only active ingredient.

Such method for treatment comprises the step of administering to a mammal a therapeutically effective amount of a composition according to the invention, preferably in the form of an oral composition such as a pill, granulate, capsule or tablet. Preferably said pill, granulate, capsule or tablet is an acid-instable pill, granulate, capsule or tablet.

In one of its embodiments, the invention of the composition as described herein is administered to a subject in need thereof at the time of or during a, preferably each, meal, snack or shot, in one or more (acid-instable) pill(s), granulate(s), capsule(s) or tablet(s). As already described the preferred acid-instable pills, granulates, capsules or tablet are typically combined with a conventional enteric coated formulation comprising enzymes such as a lipase, a phospholipase, an amylase, and/or a neutral, slightly alkaline protease. Even more preferred, the enteric coated pill, granulate, capsule or tablet comprises a lipase alone or a lipase, a phospholipase and/or an amylase and is preferably free from a neutral to slightly alkaline protease.

The invention further provides use of at least one protease with a pH optimum below pH 5.0 or which is active under acidic conditions, preferably in the stomach, and wherein said protease is further active in the presence of pepsin in the absence of other proteases for digesting proteins into substances capable of crossing the intestinal cell wall. Examples of suitable proteases are TPAP, a proline-specific endoprotease and/or a carboxypeptidase. Preferably such a use is performed in an in vitro setting, i.e. in a model system to test the efficacy of the mentioned proteases or any combination thereof.

The invention will be explained in more detail in the following detailed description which does not limit the invention.

### EXPERIMENTAL PART

### Materials & methods

Enzymes used were obtained from Sigma or produced in house. Overproduction and chromatographic purification of the proline specific endoprotease from Aspergillus niger was accomplished as described in WO 02/45524. Overproduction and chromatographic purification of the tripeptidyl aminopeptidase A (gene 12) was accomplished as described in WO 03/102195. Carboxypeptidase CPD-1 was obtained by overexpressing gene 51 specified in WO 02/068623 in A. niger. After recovery as fermentation liquid and concentration, its chromatographic purification took place as described by Dal Degan et al. in Applied and Environmental Microbiology, July 1992, 2144-2152.

Chromogenic peptide substrates were obtained either from Pepscan Systems (Lelystad, The Netherlands) or from Bachem, (Bubendorff, Switzerland).

All materials used for SDS-PAGE and staining were purchased from Invitrogen (Carlsbad, Calif., US). Samples were prepared using SDS buffer according to manufacturer's instructions and separated on 12% Bis-Tris gels using MES-SDS buffer system according to manufacturers instructions. Staining was performed using Simply Blue Safe Stain (Collodial Coomassie G250).

Quantisation Free Amino Acids

To quantify the level of free amino acids in the supernatants of the casein hydrolysates prepared according the procedure outlined in Example 4, the following method was used. By applying the conventional UV detection for monitoring free amino acids after pre-column AccQ-Tag derivatization, a serious interference with small peptides occurs. In 2006, Waters (Milford Mass., US) introduced the AccQ-Tag®ᵤₗₜᵣₐ method. In this method, 6-aminoquinolyl-N-hydroxysuccinimidyl carbamate (AQC) is used as a reagent for the quantitative conversion of primary and secondary amino acids into the corresponding carbamide derivatives. In our quantitative UPLC/tandem MS based method, pre-column AccQ-Tag®ᵤₗₜᵣₐ ultra derivatization circumvents ion suppression effects from co-eluting endogeneous dipeptides. LC-MS/MS was applied for monitoring diagnostic ions of the free amino acid derivatives, i.e. the protonated carbamide derivative and one daughter ion (m/z 171), that result from the loss of the amino acid from the corresponding carbamide derivative. By monitoring the above-mentioned MS/MS transition, free amino acids are selectively monitored, also in the presence of peptides from the hydrolysate.

To measure the free amino acids in the supernatants of the casein hydrolysates prepared according to Example 4, the following protocol for sample preparation and derivatization was used.
Dissolve 40-300 mg of protein hydrolysate in 50 ml 0.1 N HCl
Transfer 100 μl of this solution into a vial
Add 100 ul of internal standard (IS) solution (containing 1 mg of each of isotope-labelled amino acids per 50 ml)
Mix vigorously
Transfer 10 μl of this sample/IS solution into a test tube
Add 70 μl borate buffer (from the Waters AccQ-Tag ultra reagent package) and mix
Add 20 μl reagent solution (from the Waters AccQ-Tag ultra reagent package) and mix immediately
Transfer the derivative solution to an injection vial
Heat the vial at 55° C. for 10 minutes and mix
Inject 1 μl into the UPLC system.

Subsequent UPLC-MS/MS analyses were performed on an Ultra high-Pressure Liquid Chromatograph (UPLC) combined with a Xevo TQ mass spectrometer from Waters under the following conditions.

UPLC

Column: Acquity UPLC BEH C₁₈, 150×2.1 mm I.D. (1.7 μm)

Flow: 0.4 ml/min
Mobile phase: Solvent A: Water/AccQ.Tag Eluent A (95:5% v/v)
   Solvent B: AccQ.Tag Eluent B
Injection volume: 1 μl partial loop with needle overfill
Injection loop: 2 μl
Column temperature: 43° C.
Tray temperature: 20° C.

Runtime: 60 min
Gradient:

| Time | Solvent | Solvent | |
|---|---|---|---|
| (min) | A (%) | B (%) | Curve |
| 0 | 99.9 | 0.1 | |
| 1.14 | 99.9 | 0.1 | linear |
| 2.00 | 98.5 | 1.5 | linear |
| 5.50 | 98.1 | 1.9 | linear |
| 6.50 | 98.0 | 2.0 | convex |
| 10.00 | 97.6 | 2.4 | linear |
| 12.00 | 96.0 | 4.0 | linear |
| 20.00 | 88.0 | 12.0 | linear |
| 35.00 | 85.0 | 15.0 | convex |
| 36.00 | 2.0 | 98.0 | linear |
| 38.00 | 2.0 | 98.0 | linear |
| 39.00 | 99.9 | 0.1 | linear |
| 60.00 | 99.9 | 0.1 | |

MS:
Ion mode: ESI positive
Capillary voltage: 1.5 V
Cone voltage: 30 V
Extractor voltage: 4.3 V
LM resolution: 3
HM resolution: 15
Ion energy: 0.5 eV
Desolvation temperature: 600° C.
Source temperature: 150° C.
Cone gas flow: 50 L/hr
Desolvation gas flow: 500 L/hr
Dwell time: 0.01 sec
Interscan delay: 0.1 sec
Interchannel delay: 0.1 sec

Quantifying Di- and Tripeptides

To roughly determine the molecular weight (MW) distribution of oligopeptides in a protein hydrolysate, usually size exclusion chromatography (GPC) is used. However, for the MW range <500 Da this technique provides insufficient information about the distribution between free amino acids, di- and tripeptides. Quantitative analysis of the di- and tripeptides by LC/MS is hampered by the fact that the proton affinity differs a factor >100 among peptides.

We determined total di- and tripeptides by quantitative LC/MS method in which we took advantage of a pre-column derivatization and exact mass analysis using a LTQ orbitrap mass spectrometer. Using the AccQ-Tag®ᵤₗₜᵣₐ derivatization technique (see above) it was possible to significantly reduce the difference in proton affinity between the various peptides. The derivatization was carried out according to the following procedure.
Prepare a 1 mg/ml protein hydrolysate solution in MilliQ
Transfer 10 μl of this solution in a test tube
Add 70 μl borate buffer (from the Waters AccQ-Tag ultra reagent package) and mix
Add 20 μl reagent solution (from the Waters AccQ-Tag ultra reagent package) and mix
Transfer the solution to an injection vial
Heat the vial at 55° C. for 10 minutes and mix
Inject a volume into the LC/MS system.
   Subsequent analyses were performed using an Accela HPLC (in high pressure Mode, coupled with a LTQ/orbitrap mass spectrometer from Thermo Electron). An Agilent SB-C18 column (1.8 um, 2.1*50 mm) was used at 55° C. for separation of the peptides, with gradient elution, starting at 96% A (0.1% Formic acid (FA)) and increasing to 30% B (0.1% FA in Acetonitrile (ACN)) in 7 minutes, directly equilibrating at 96% A for three minutes. The flow-rate was 0.4 ml/min, and the injection volume was 5 ul. Samples were stored at 4° C. upon analysis. The LTQ/orbitrap was operated in ESI/pos mode and scanning from m/z 100-1500 at a resolution of 60000. The mass accuracy was kept <2 ppm by daily calibrating the orbitrap.

Using a wide variety of dipeptide standards, first the deviation of the proton affinity (expressed in peak area) was determined before/after derivatization. A total of 20 different dipeptides, selected on the basis of their hydrophobicities, were used and after subsequent derivatization and LC/MS analysis the peak area per pMole dipeptide was calculated. The analysis was performed in 5-fold. Typically for the 20 dipeptides a RSD of <30% was obtained whereas for the non-derivatized dipeptides a RSD of >80% was obtained. On the basis of a similar measurement of 20 different derivatized tripeptides a RSD of 40% was obtained. The di- and tri-peptides present in the casein hydrolysate were determined in MS mode using accurate mass detection.

To calculate the absolute amount of total di- tri-peptides in the casein hydrolysate prepared according to the procedure described in Example 4, known amounts of some di- and tri-peptides (which do not occur in the amino acid sequence of the caseine) were added to the sample as standard. For quantification automatically all theoretical exact masses were plotted as ion chromatograms. After integration all peak areas of the individual di- and tripeptides were summed, whereas also after integration all peak areas of the standard di- and tripeptides were summed. The ratio of these areas results in the quantification of total di- and tripeptides.

### Example 1

The pH Profiles of Various Proteases as Obtained from A. niger

WO 02/068623 and WO 02/45524 specify various proteases that are encoded by the food grade microorganism Aspergillus niger. Genes 10, 12 of WO 02/068623 encode two highly homologous but slightly different tripeptidyl aminopeptidases; gene 51 encodes carboxypeptidase CPD-I. In WO 02/45524 the sequence of a proline-specific endoproteases is provided. All four proteases were obtained in industrially relevant quantities by overexpression of the four genes an A. niger host cell using methods specified in the prior art. As all four proteases are efficiently secreted by the A. niger host cell, recovery of the crude enzymes is relatively simple. An example of the chromatographic purification of the two tripeptidyl aminopeptidases is provided in WO 03/102195, an example of the chromatographic purification of the proline-specific endoprotease in WO 02/45524 and an example of the chromatographic purification of the carboxypeptidase in Applied and Environmental Microbiology, July 1992, 2144-2152.

The pH profiles of the various chromatographically purified enzymes were obtained using different chromogenic peptides. The pH profiles of the two tripeptidyl aminopeptidases were established using peptide Ala-Ala-Phe-pNA, the pH profile of the proline-specific endoprotease was determined using peptide Z-Gly-Pro-pNA and the pH profile of the carboxypeptidase was determined using peptide FA-Phe-Ala. “Z” represents a benzyloxycarbonyl group, “pNA” the chromophore para-nitroanilide and “FA” the chromophore 3-(2-furyl)acryloyl. Stock solutions of the chromogenic substrates were prepared in methanol (FA-Phe-Ala) or in DMSO (pNA substrates) and diluted 100× in the desired aqueous buffer. Buffers ranging from pH 2.0 to 7.0 were prepared using 0.1 mol/l citrate, buffers ranging from pH 6.0 to 9.0 were prepared using 0.1 mol/l tris and buffers ranging from pH 8.0 to 12.0 were made using 0.2 mol/l glycine. The required pH values were adjusted using either HCl or NaOH. Using pNA substrates the increase in absorbance at 410 nm was used as a measure for enzyme activity, with FA-Phe-Ala the decrease in the absorbance was followed at 337 nm. Incubations were carried out for 10 minutes.

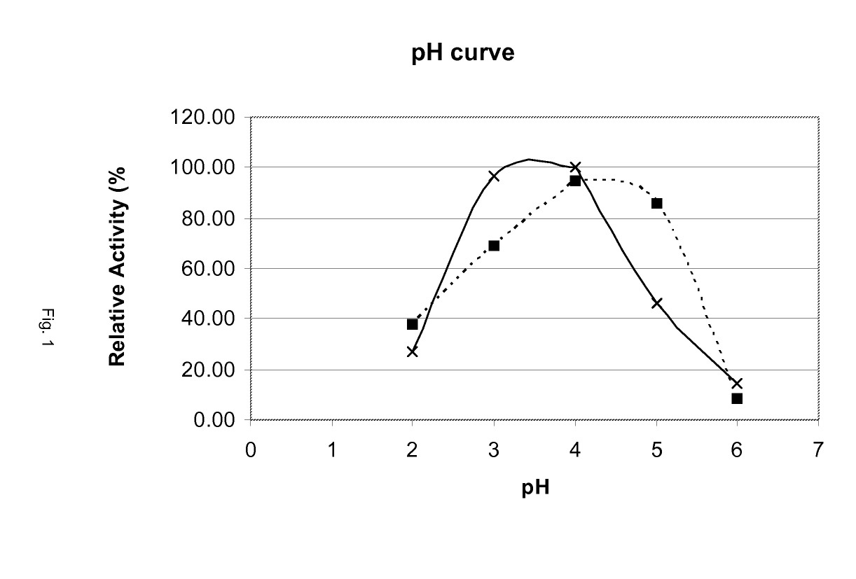

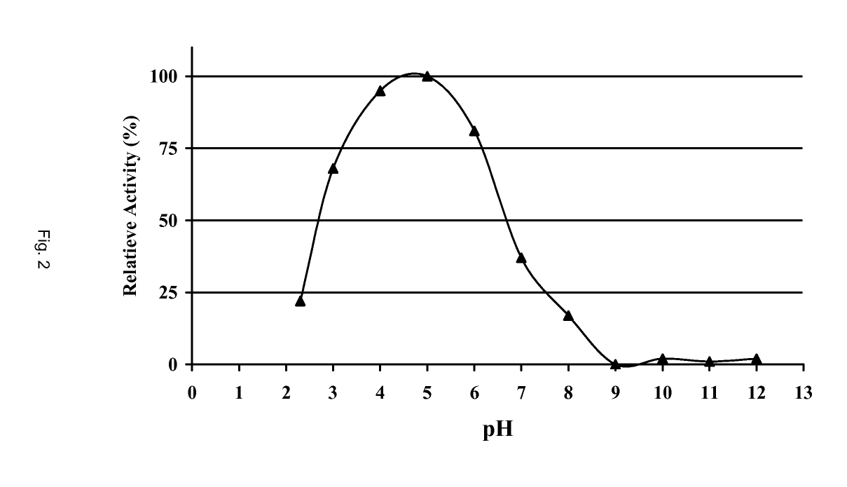

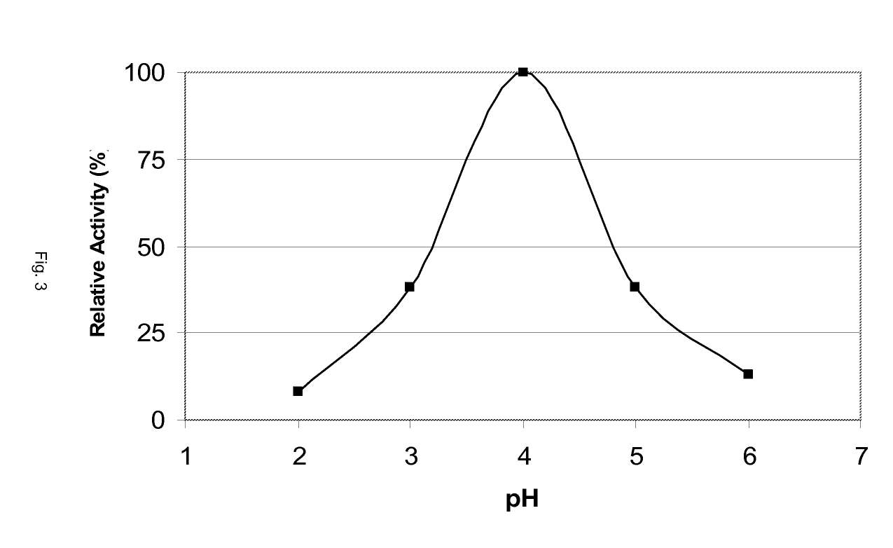

The different pH profiles as obtained for the four enzymes are shown in FIGS. 1 to 3.

According to the data obtained all four proteases have a pH optimum well below pH 5.0. At pH values higher than 6.0 both tripeptidyl aminopeptidases and carboxypeptidase CPD-1 show residual enzyme activities of less than 10% of their activity around pH 4.0. At a pH value of 6.0 the proline-specific protease has a residual enzyme activity of about 75% of its activity around pH 4.0. At pH 7.0 the residual activity is 30% of its maximum.

### Example 2

### Stabilities of the A. niger Proline Specific Endoprotease Under Conditions as Present in the Stomach

Prerequisite for a successful enzyme therapy according to the present application is an efficient degradation of dietary proteinaceous material in the stomach. This requires that the exogeneous protease is optimally active in the stomach, i.e. at low pH values and in the presence of the gastric protease pepsin. To evaluate the activity of the A. niger derived proline specific protease under such “stomach-like” conditions, we assayed its residual activity after an incubation at 37 degrees C. for different time periods under different pH conditions and in the presence and absence of pepsin. Citrate/HCl buffers of 0.2 mol/l were used for obtaining the required acid pH conditions. The dosage of the A. niger derived enzyme was 1.5 units/ml and pepsin (from porcine stomach mucosa, 2331 U/mg, Sigma P-7012) was added in a concentration of 180 microgram/ml. Pepstatin (Sigma) was added after sampling in a concentration of 1.67 microgram/ml in order to inactivate the pepsin. Under these conditions pepstatin had no inhibitory effect on the proline specific protease. Residual activities of the proline specific protease were measured kinetically at 405 nm using the synthetic substrates Ala-Ala-Pro-pNA (Bachem, Switzerland). To that end 200 μL substrate solution (1.5 mmol/l Ala-Ala-Pro-pNA in a 0.05 mol/l acetic acid buffer pH 4.0) was mixed with a 50 microliter (prediluted 10 to 100×) of the acid/pepsin treated sample in MTP wells. Absorbance was measured kinetically for 10 min at 405 nm at 30° C. making use of a TECAN Genios MTP Reader (Salzburg, Vienna).

The results depicted in Table 1 show that the proline specific protease maintains its full activity at pH values as low as pH 2 and even in combination with pepsin. This finding implies that the enzyme will be fully active during its presence in the stomach and will be ideally suited for degrading proline rich peptides in the stomach.

TABLE 1
Residual enzyme activity of the A. niger endoprotease after
various incubation periods under stomach-like conditions

| | Residual enzyme activity of |
|---|---|
| | the A. niger endoprotease |
| Incubation | after: |

| | | | | |
|---|---|---|---|---|
| Conditions | 15 | 30 | 60 | 120 |

| pH | Pepsin present | mins | mins | mins | mins |
|---|---|---|---|---|---|
| 2 | No | + | + | + | + |
| | Yes | + | + | + | + |
| 3 | No | + | + | + | + |
| | Yes | + | + | + | + |
| 4 | No | + | + | + | + |
| | Yes | + | + | + | + |

+ means residual activity present if tested under conditions optimal for the enzyme,
− means no residual activity present if tested under conditions optimal for the enzyme.

### Example 3

### Tabletting the Proteases According to the Invention

Starting from the powdered protease, an enzyme containing tablet suitable for oral intake can be prepared according to the following protocol. To 2.80 g Polyplasdone XL10 (Crospovidone), add 180.56 g Avicel pH 302 microcrystalline cellulose and push through a 1 mm sieve. Then add 95.24 g of enzyme powder and mix for 10 minutes with a tumbler mixer. Add 1.4 g Mg-stearate and mix again for 2 min. The resulting tablet mixture is then compressed to tablets on a single punch press:

Tablet press: Comprex II
Punch: oblong, 22 mm×9 mm
Compression force: 20 kN
The tablets obtained weigh approximately 1400 milligrams and incorporate 480 mg of the powdered protease.

### Example 4

### Tripeptide and Amino Acid Production Under Simulated Stomach Conditions

According to the present invention an oral protease acting in the stomach in conjunction with the pepsin present, leads to the production of small peptides and free amino acids which can be readily transported over the intestinal wall to end up in the blood circulation. To illustrate the value of this concept, the following in vitro experiment was carried out.

Sodium caseinate (Tatua E 5146) was suspended in water and the pH was adjusted to 4.0 using HCl. Precipitated casein was homogenised using an Ultra Turrax. Then, in five different incubations, pure enzymes (dissolved in 20 millimol/l acetate buffer pH 4.0) were added according to the following scheme.
1) 2.5 mg/ml caseinate suspension without enzyme addition (reference)
2) 2.5 mg/ml caseinate suspension plus 0.25 mg/ml of pepsin from porcine stomach mucosa (Sigma P-6887 3200-4500 U/mg protein lot #128K7354)
3) 2.5 mg/ml caseinate suspension plus 0.25 mg/ml of pepsin plus 0.25 mg/ml TPAP-A
4) 2.5 mg/ml caseinate suspension plus 0.25 mg/ml of pepsin plus 0.25 mg/ml TPAP-A+0.25 mg/ml carboxypeptidase CPD-1
5) 2.5 mg/ml caseinate suspension plus 0.25 mg/ml TPAP-A
   After an incubation for 45 and for 90 minutes at 37 degrees C. with mild shaking, the following visual observations of the various incubations could be made (Table 2).

TABLE 2
Visual impression of the various enzyme
incubations under stomach-like conditions

Visual impression after

| Incubation | Enzymes added | 45″ | 90″ |
|---|---|---|---|
| 1 | none | turbid | turbid |
| 2 | Pepsin | turbid | clear |
| 3 | Pepsin + TPAP | Almost clear | clear |
| 4 | Pepsin + TPAP + CPD | Almost clear | clear |
| 5 | TPAP | turbid | turbid |

These observations strongly suggest that in the incubations in which next to pepsin TPAP-A is present, the caseine hydrolysis proceeds more efficiently than in incubations with only pepsin or only TPAP-A present.

After finalising these visual observations, i.e. immediately after the 90 minutes of incubation, all enzymatic reactions were terminated by heating for 10 minutes at 95 degrees C. After centrifugation the clear supernatant was recovered (i.e. from the clear solutions specified in Table 2 all casein was recovered, from the turbid solutions only part of the casein was recovered in the supernatant) and subjected to a quantitative measurement of the di- and tripeptides (Table 3) and the free amino acids (Table 4) according to procedures specified in the Materials & Methods section. Free amino acids as well as di- and tri-peptides represent nutritional units that are readily absorbable through the intestinal wall, i.e. without a further degradation by pancreatic enzymes. Thus, the sum of free amino acids, di- and tripeptides represents the fraction of the soluble casein that was converted under stomach-like conditions into readily absorbable units by the proteolytic enzymes present. As can be seen from the data presented in Table 5, under the influence of TPAP and especially TPAP combined with carboxypeptidase CPD, high proportions of the casein substrate present are converted into such readily absorbable units.

TABLE 3
Levels of di- and tri-peptides established in the various
enzyme incubations under stomach-like conditions

| | | Weight % of soluble casein | |
|---|---|---|---|
| | | present in the form of: | |
| Incubation | Enzymes added | dipeptides | tripeptides |
| 1 | none | 0.06 | 0.14 |
| 2 | Pepsin | 1.0 | 8.6 |
| 3 | Pepsin + TPAP | 4.4 | 20.2 |
| 4 | Pepsin + TPAP + CPD | 6.9 | 12.9 |
| 5 | TPAP | 0.16 | 4.1 |

TABLE 4
Levels of free amino acids established in the various
enzyme incubations under stomach-like conditions

| | | Weight % of soluble casein |
|---|---|---|
| | | present in the form of free |
| Incubation | Enzymes added | amino acids |
| 1 | none | 0.16 |
| 2 | Pepsin | 0.94 |
| 3 | Pepsin + TPAP | 8.05 |
| 4 | Pepsin + TPAP + CPD | 41.27 |
| 5 | TPAP | 0.89 |

TABLE 5
Weight percentages of casein converted under stomach-
like conditions into readily absorbable units

| | | Weight % of soluble casein present |
|---|---|---|
| | | in the form of either free amino acids, |
| Incubation | Enzymes added | di- or tri-peptides |
| 1 | none | 0.4 |
| 2 | Pepsin | 10.5 |
| 3 | Pepsin + TPAP | 32.7 |
| 4 | Pepsin + TPAP + CPD | 61.1 |
| 5 | TPAP | 5.2 |

1. A pharmaceutical or nutritional composition for the treatment of pancreatic enzyme insufficiency, pancreatitis or cystic fibrosis comprising at least one protease with a pH optimum below pH 5.0 and wherein said protease is active in the presence of pepsin.

2. A pharmaceutical or nutritional composition for the treatment of pancreatic enzyme insufficiency, pancreatitis or cystic fibrosis comprising at least one protease which is active at acidic pH, optionally in the stomach, and wherein said protease is active in the presence of pepsin.

3. A composition according to claim 1, wherein said protease has at pH 6.0 or higher an activity of less than 50%, preferably less than 40% and even more preferably less than 30% compared to its activity at pH 4.0.

4. A composition according to claim 1, wherein said protease is a tripeptidase optionally said tripeptidase is a tripeptidyl aminopeptidase or peptidyl-tripeptidase.

5. A composition according to claim 1, further comprising a second protease wherein optionally said second protease is a proline-specific endoprotease and/or a carboxypeptidase.

6. A composition according to claim 1, wherein said protease(s) is/are present in an acid-instable pill, granulate, capsule or tablet.

7. An acid-instable pill, granulate, capsule or tablet for the treatment of pancreatic enzyme insufficiency, pancreatitis or cystic fibrosis comprising at least one protease with a pH optimum below pH 5.0 and said protease is further active in the presence of pepsin.

8. An acid-instable pill, granulate, capsule or tablet for the treatment of pancreatic enzyme insufficiency, pancreatitis or cystic fibrosis comprising at least one protease which is active at acidic pH, optionally in the stomach, and said protease is further active in the presence of pepsin.

9. An acid-instable pill, granulate, capsule or tablet according to claim 7 wherein said at least one protease is a tripeptidase, optionally a tripeptidylaminopeptidase.

10. A method for preparing an acid-instable pill, granulate, capsule or tablet for the treatment of pancreatic enzyme insufficiency, pancreatitis or cystic fibrosis according to claim 7 comprising obtaining at least one protease with a pH optimum below pH 5.0 and said protease is active in the presence of pepsin and preparing an acid-instable pill, granulate, capsule or tablet comprising the protease.

11. A method for preparing an acid-instable pill, granulate, capsule or tablet for the treatment of pancreatic enzyme insufficiency, pancreatitis or cystic fibrosis according to claim 7 comprising obtaining at least one protease which is active at acidic pH, optionally in the stomach, and said protease is active in the presence of pepsin and preparing an acid-instable pill, granulate, capsule or tablet comprising the protease.

12. A protease with a pH optimum below pH 5.0 or which is active at acidic pH, optionally in the stomach, and wherein said protease is further active in the presence of pepsin for the manufacture of a medicament for the treatment of pancreatic enzyme insufficiency, pancreatitis or cystic fibrosis optionally said protease is a tripeptidase.

13. A protease according to claim 12, wherein said at least one protease is present in an acid instable pill, granulate, capsule or tablet.

14. A protease according to claim 13, wherein said acid instable pill, granulate, capsule or tablet further comprises a proline-specific endoprotease and/or a carboxypeptidase.

15. A method for treating malabsorption in a mammal comprising administering to said mammal a therapeutically effective amount of a composition according to claim 1.

16. A method for treating pancreatic enzyme insufficiency, pancreatitis or cystic fibrosis in a mammal comprising administering to said mammal a therapeutically effective amount of a composition according to claim 1.

17. A method for supplementing enzymes in a mammal for the treatment of pancreatic enzyme insufficiency, pancreatitis or cystic fibrosis comprising administering to said mammal a therapeutically effective amount of a composition according to claim 1.

18. A method according to claim 15, wherein said composition is in the form of a pill, granulate, capsule or tablet.

19. A method according to claim 15, wherein said mammal suffers from cystic fibrosis.

20. A method according to claim 15, wherein the composition is administered to said mammal with a meal, snack or shot.

21. A protease which is active at pH 5 or lower for the treatment of pancreatic enzyme insufficiency, pancreatitis or cystic fibrosis and wherein said protease is further active in the presence of pepsin, in the absence of other proteases for digesting proteins into substances capable of crossing the intestinal cell wall.
